(19) United States
(12) Patent Application Publication
(10) Pub. No.: US 2010/0010044 A1
(43) Pub. Date: Jan. 14, 2010

(54) NOVEL CRYSTALLINE FORM OF 2-[4-(4-FLUORO-BENZYL)-PIPERIDINE-1-YL]-2-OXO-N-(2-OXO-2,3-DIHYDRO-BENZOXAZOL-6-YL)-ACETAMIDE

(75) Inventors: Alphonso HIGUERA, Farmingdale, NY (US); Haijian ZHU, Smithtown, NY (US); Andreas GRILL, Hauppauge, NY (US)

Correspondence Address: Forest Laboratories, Inc.;Attn: Charles S. Ryan, 48 Mall Drive, Commack, NY, 11725, US

(73) Assignee: Forest Laboratories Holdings Limited, Hamilton (BM)

(21) Appl. No.: 12/499,243

(22) Filed: Jul. 8, 2009

Related U.S. Application Data
(60) Provisional application No. 61/078,916, filed on Jul. 8, 2008.

(51) Int. Cl. A61K 31/454; C07D 413/12; A61P 25/00; A61P 25/18; A61P 25/24

(52) U.S. Cl. 514/321; 546/198

(57) ABSTRACT

The present invention relates to a novel crystalline form of 2-[4-(4-fluoro-benzyl)-piperidine-1-yl]-2-oxo-N-(2-oxo-2,3-dihydro-benzoxazol-6-yl)-acetamide. Processes for the preparation of this form, compositions containing the form, and methods of use thereof are also described.

### FIELD OF THE INVENTION

The present invention relates to a novel crystalline form of 2-[4-(4-fluoro-benzyl)-piperidine-1-yl]-2-oxo-N-(2-oxo-2,3-dihydro-benzoxazol-6-yl)-acetamide. Processes for the preparation of this form, compositions containing the form, and methods of use thereof are also described.

### BACKGROUND OF THE INVENTION

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. A new polymorphic form of radiprodil has now been discovered.

### SUMMARY OF THE INVENTION

In one embodiment, the present invention relates to a crystalline form of 2-[4-(4-fluoro-benzyl)-piperidine-1-yl]-2-oxo-N-(2-oxo-2,3-dihydro-benzoxazole-6-yl)-acetamide as represented by formula (I) having an X-ray powder diffraction pattern comprising characteristic peaks at about 6.4, about 13.7, and about 25.8±0.2 degrees 2θ.

In another embodiment, the present invention relates to a process of preparing a crystalline form of 2-[4-(4-fluoro-benzyl)-piperidine-1-yl]-2-oxo-N-(2-oxo-2,3-dihydro-benzoxazole-6-yl)-acetamide comprising:

(i) forming a mixture of 2-[4-(4-fluoro-benzyl)-piperidine-1-yl]-2-oxo-N-(2-oxo-2,3-dihydro-benzoxazole-6-yl)-acetamide dihydrate, water and acetone;

(ii) maintaining the mixture for a period of time, and

(iii) optionally isolating the crystalline form.

In another embodiment, the present invention relates to a pharmaceutical composition comprising the crystalline form of 2-[4-(4-fluoro-benzyl)-piperidine-1-yl]-2-oxo-N-(2-oxo-2,3-dihydro-benzoxazole-6-yl)-acetamide as represented by formula (I) having an X-ray powder diffraction pattern comprising characteristic peaks at about 6.4, about 13.7, and about 25.8±0.2 degrees 2θ.

In yet another embodiment, the present invention relates to a method for treating and/or preventing a condition which requires modulation of an NMDA receptor comprising administering to a patient in need thereof, an effective amount of the crystalline form of 2-[4-(4-fluoro-benzyl)-piperidine-1-yl]-2-oxo-N-(2-oxo-2,3-dihydro-benzoxazole-6-yl)-acetamide as represented by formula (I) having an X-ray powder diffraction pattern comprising characteristic peaks at about 6.4, about 13.7, and about 25.8±0.2 degrees 2θ.

### BRIEF DESCRIPTION OF THE FIGURES

FIG. 1 shows the X-ray powder diffraction pattern of Form C of 2-[4-(4-fluoro-benzyl)-piperidine-1-yl]-2-oxo-N-(2-oxo-2,3-dihydro-benzoxazol-6-yl)-acetamide (radiprodil).

FIG. 2 shows the Fourier Transform infrared spectrum of Form C of 2-[4-(4-fluoro-benzyl)-piperidine-1-yl]-2-oxo-N-(2-oxo-2,3-dihydro-benzoxazol-6-yl)-acetamide (radiprodil).

FIG. 3 shows the Raman spectrum of Form C of 2-[4-(4-fluoro-benzyl)-piperidine-1-yl]-2-oxo-N-(2-oxo-2,3-dihydro-benzoxazol-6-yl)-acetamide (radiprodil).

FIG. 4 shows the differential scanning calorimetry trace for Form C of 2-[4-(4-fluoro-benzyl)-piperidine-1-yl]-2-oxo-N-(2-oxo-2,3-dihydro-benzoxazol-6-yl)-acetamide (radiprodil).

FIG. 5 shows the thermogravimetric analysis for Form C of 2-[4-(4-fluoro-benzyl)-piperidine-1-yl]-2-oxo-N-(2-oxo-2,3-dihydro-benzoxazol-6-yl)-acetamide (radiprodil).

### DETAILED DESCRIPTION OF THE INVENTION

One embodiment of the present invention relates to a crystalline form of 2-[4-(4-fluoro-benzyl)-piperidine-1-yl]-2-oxo-N-(2-oxo-2,3-dihydro-benzoxazol-6-yl)-acetamide. U.S. Publication no. 2004/0157886 discloses novel piperidine derivatives as antagonists of NMDA receptors. All formulations cited in the U.S. Publication are hereby incorporated by reference in their entirety.

One particular compound disclosed therein, 2-[4-(4-fluoro-benzyl)-piperidine-1-yl]-2-oxo-N-(2-oxo-2,3-dihydro-benzoxazol-6-yl)-acetamide, also known as radiprodil, is a highly effective NR2B subtype selective antagonist of NMDA receptors. The structural formula of radiprodil is shown below in formula (I).

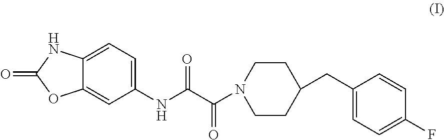

Another embodiment of the present invention relates to a crystalline form of 2-[4-(4-fluoro-benzyl)-piperidine-1-yl]-2-oxo-N-(2-oxo-2,3-dihydro-benzoxazol-6-yl)-acetamide as represented by formula (I), wherein the crystalline form is Form C.

In yet another embodiment the crystalline Form C can be anhydrous.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value.

The term “substantially pure” means a compound having a purity greater then, e.g., about 90% by weight, for example, greater than about 91% by weight, greater than about 92% by weight, greater than about 93% by weight, greater than about 94% by weight, greater than about 95% by weight, greater than about 96% by weight, greater than about 97% by weight, greater than about 97.5% by weight, greater than about 98% by weight, greater than about 99% by weight, greater than about 99.5% by weight, or greater than about 99.9% by weight.

The term “treating” means to relieve, alleviate, delay, reduce, reverse, improve or prevent at least one symptom of a condition in a subject. The term “treating” may also mean to arrest, delay the onset (i.e., the period prior to clinical manifestation of a disease) and/or reduce the risk of developing or worsening a condition.

An “effective amount” means the amount of the crystalline form of the present invention that, when administered to a patient (e.g., a mammal) for treating a disease, is sufficient to effect such treatment for the disease, or an amount of a compound that is sufficient for modulating an NMDA receptor (e.g., an NR2B selective NMDA receptor) to achieve the objectives of the invention. The “effective amount” will vary depending on the compound, the disease and its severity and the age, weight, etc., of the patient to be treated.

In one embodiment, the present invention relates to a crystalline form of 2-[4-(4-fluoro-benzyl)-piperidine-1-yl]-2-oxo-N-(2-oxo-2,3-dihydro-benzoxazole-6-yl)-acetamide having an X-ray powder diffraction pattern comprising one or more peaks as provided in Table 1.

TABLE 1
Form C

| 2θ (°) | d-spacing (\acute{Å}) |
|---|---|
| 6.4 | 13.9 |
| 8.0 | 11.1 |
| 9.6 | 9.2 |
| 11.5 | 7.7 |
| 12.8 | 6.9 |
| 13.7 | 6.5 |
| 15.4 | 5.8 |
| 16.0 | 5.5 |
| 17.0 | 5.2 |
| 17.3 | 5.1 |
| 18.9 | 4.7 |
| 19.3 | 4.6 |
| 19.8 | 4.5 |
| 20.4 | 4.3 |
| 21.2 | 4.2 |
| 21.7 | 4.1 |
| 24.1 | 3.7 |
| 24.6 | 3.6 |
| 25.8 | 3.5 |
| 26.3 | 3.4 |
| 27.1 | 3.3 |
| 27.5 | 3.2 |
| 28.7 | 3.1 |
| 29.0 | 3.1 |
| 29.7 | 3.0 |
| 30.5 | 2.9 |
| 32.4 | 2.8 |
| 32.8 | 2.7 |
| 33.3 | 2.7 |
| 33.5 | 2.7 |
| 34.7 | 2.6 |
| 35.3 | 2.5 |
| 35.7 | 2.5 |
| 36.7 | 2.4 |
| 37.3 | 2.4 |
| 38.5 | 2.3 |
| 39.3 | 2.3 |

In another embodiment, the present invention relates to a crystalline form of 2-[4-(4-fluoro-benzyl)-piperidine-1-yl]-2-oxo-N-(2-oxo-2,3-dihydro-benzoxazole-6-yl)-acetamide having an X-ray powder diffraction pattern comprising one or more peaks at about 6.4, about 8.0, about 13.7, about 19.8, about 21.7, about 24.1, and about 25.8±0.2 degrees 2θ.

In yet another embodiment, the present invention relates to a crystalline form of 2-[4-(4-fluoro-benzyl)-piperidine-1-yl]-2-oxo-N-(2-oxo-2,3-dihydro-benzoxazole-6-yl)-acetamide having an X-ray powder diffraction pattern comprising one or more peaks at about 6.4, about 13.7, and about 25.8±0.2 degrees 2θ.

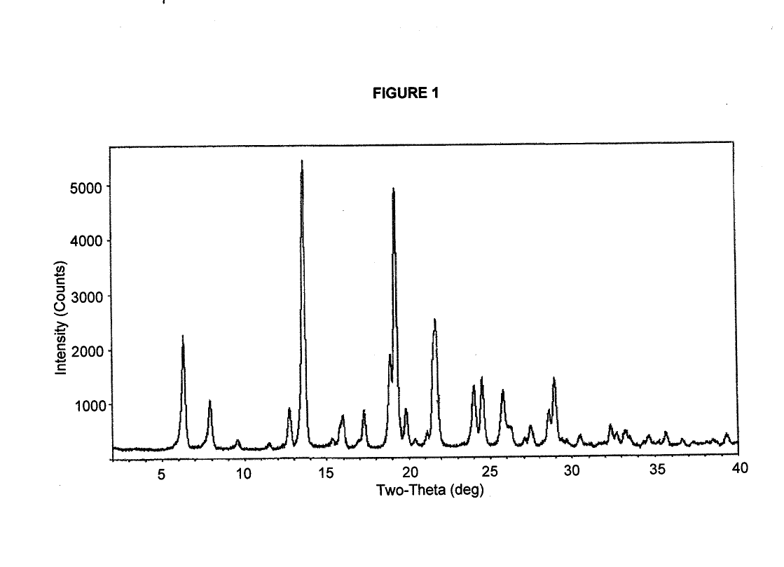

In a further embodiment, Form C of 2-[4-(4-fluoro-benzyl)-piperidine-1-yl]-2-oxo-N-(2-oxo-2,3-dihydro-benzoxazol-6-yl)-acetamide is characterized by a X-ray powder diffraction pattern substantially as shown in FIG. 1. With respect to the term “substantially,” one skilled in the art would understand that the relative intensities of the peaks can vary, depending upon the sample preparation technique, the sample mounting procedure and the particular instrument employed. Moreover, instrument variation and other factors can affect the 2θ values. Therefore, the XRD peak assignments can vary by plus or minus about 0.2 degrees 2θ.

In yet another embodiment, Form C of 2-[4-(4-fluoro-benzyl)-piperidine-1-yl]-2-oxo-N-(2-oxo-2,3-dihydro-benzoxazol-6-yl)-acetamide is characterized by an X-ray diffraction pattern further comprising d spacing peaks at about 13.9, about 6.5, and about 3.5 Å.

One skilled in the art will understand that 2θ values may change depending on wavelength λ of the X-rays, even as the d-spacing values remain constant.

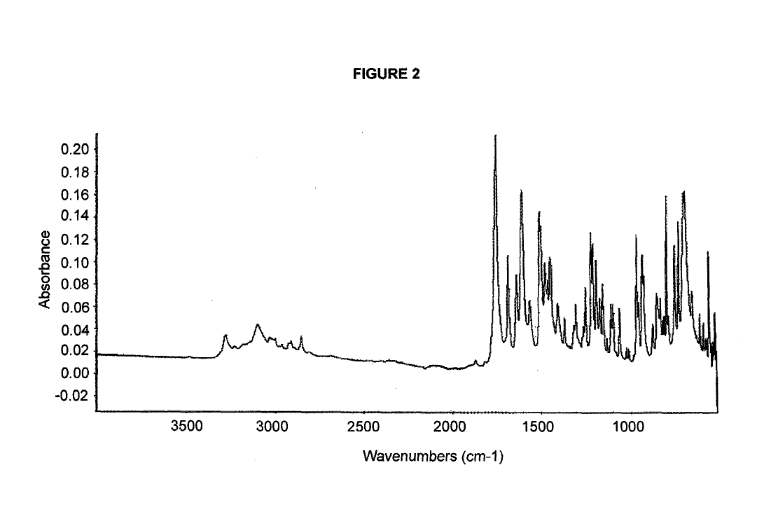

Form C of 2-[4-(4-fluoro-benzyl)-piperidine-1-yl]-2-oxo-N-(2-oxo-2,3-dihydro-benzoxazol-6-yl)-acetamide can also be identified by its Fourier Transform infrared spectrum, which is shown in FIG. 2.

In another embodiment, the present invention provides a crystalline form of 2-[4-(4-fluoro-benzyl)-piperidine-1-yl]-2-oxo-N-(2-oxo-2,3-dihydro-benzoxazol-6-yl)-acetamide (Form C) which is characterized by a Fourier Transform infrared spectrum comprising characteristic peaks at about 3278, about 3106, about 2846, about 1683 and about 1560 cm⁻¹.

In yet another embodiment, Form C of 2-[4-(4-fluoro-benzyl)-piperidine-1-yl]-2-oxo-N-(2-oxo-2,3-dihydro-benzoxazol-6-yl)-acetamide is characterized by a Fourier Transform infrared spectrum substantially as shown in FIG. 2.

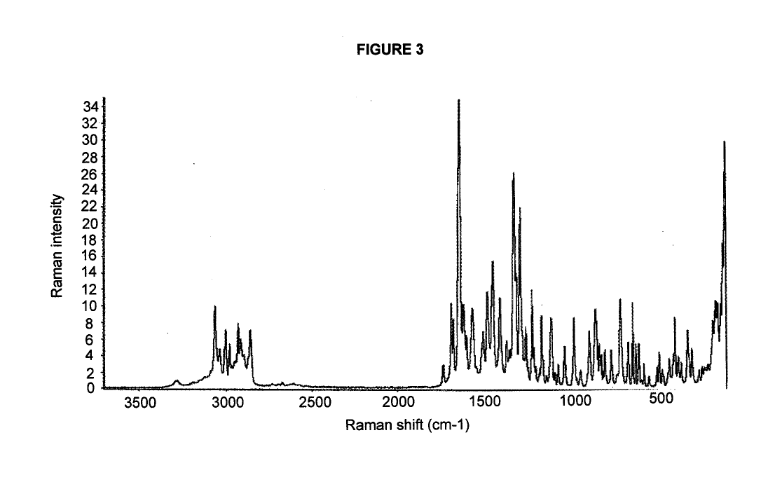

Form C of 2-[4-(4-fluoro-benzyl)-piperidine-1-yl]-2-oxo-N-(2-oxo-2,3-dihydro-benzoxazol-6-yl)-acetamide can also be identified by its Raman spectrum, which is shown in FIG. 3.

In another embodiment, the present invention provides a crystalline form of 2-[4-(4-fluoro-benzyl)-piperidine-1-yl]-2-oxo-N-(2-oxo-2,3-dihydro-benzoxazol-6-yl)-acetamide (Form C) which is characterized by a Raman spectrum comprising characteristic peaks at about 3280, about 3030, about 1730 and about 1570 cm⁻¹.

In yet another embodiment, Form C of 2-[4-(4-fluoro-benzyl)-piperidine-1-yl]-2-oxo-N-(2-oxo-2,3-dihydro-benzoxazol-6-yl)-acetamide is characterized by a Raman spectrum substantially as shown in FIG. 3.

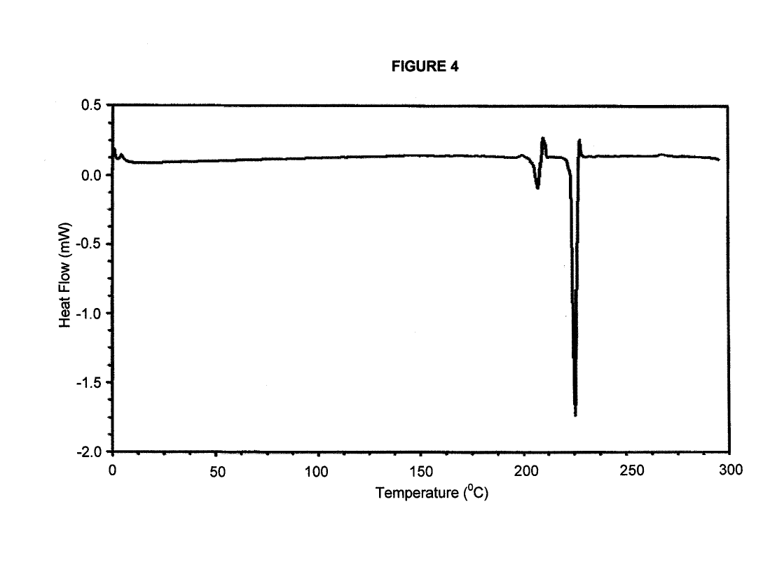

In another embodiment, Form C of 2-[4-(4-fluoro-benzyl)-piperidine-1-yl]-2-oxo-N-(2-oxo-2,3-dihydro-benzoxazol-6-yl)-acetamide can also be identified by its characteristic differential scanning calorimetry (DSC) trace, such as shown in FIG. 4. In yet another embodiment, Form C is characterized by a DSC trace showing a first endothermic transition with an onset at about 205° C. and a second endothermic transition with an onset at about 224° C.

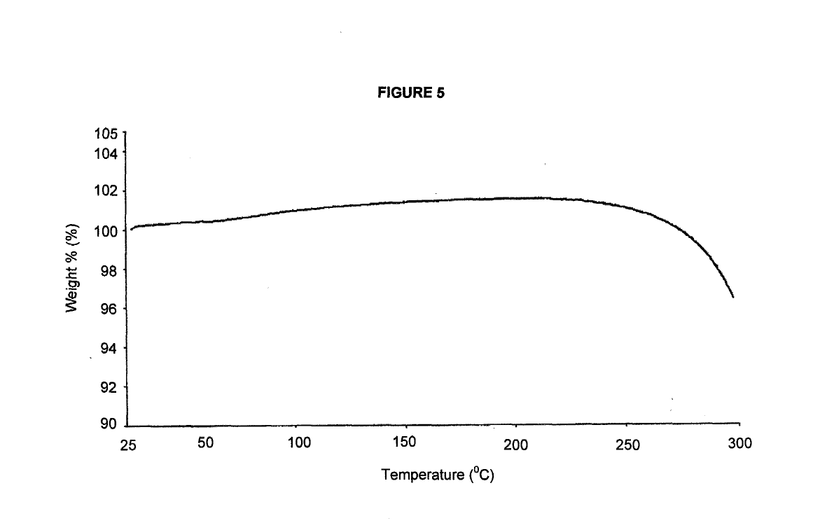

The thermogravimetric analysis (TGA) trace for Form C of 2-[4-(4-fluoro-benzyl)-piperidine-1-yl]-2-oxo-N-(2-oxo-2,3-dihydro-benzoxazol-6-yl)-acetamide is shown in FIG. 5.

The present invention also provides processes for preparing Form C of 2-[4-(4-fluoro-benzyl)-piperidine-1-yl]-2-oxo-N-(2-oxo-2,3-dihydro-benzoxazol-6-yl)-acetamide.

In one embodiment, Form C may be prepared a process that comprises (i) forming a mixture of 2-[4-(4-fluoro-benzyl)-piperidine-1-yl]-2-oxo-N-(2-oxo-2,3-dihydro-benzoxazol-6-yl)-acetamide dihydrate, water and acetone; (ii) maintaining the mixture for a period of time, and (iii) optionally isolating the crystalline form.

In one embodiment, the ratio of water:acetone is from about 10:90 to about 30:70 v/v.; from about 15:85 to about 25:75; from about 20:80 to about 25:75; inclusive of all ranges and sub-ranges therein. In another embodiment, the mixture of 2-[4-(4-fluoro-benzyl)-piperidine-1-yl]-2-oxo-N-(2-oxo-2,3-dihydro-benzoxazol-6-yl)-acetamide and water:acetone forms a slurry. In a further embodiment, the mixture is maintained at room temperature in step (ii). In additional embodiments, the period of time in step (ii) is about 1 week, about 2 weeks, about 3 weeks, about 1 month, inclusive of all ranges and sub-ranges therein.

The crystal forms may be dried. For example, drying is carried out at atmospheric pressure (e.g., by allowing the solvent to evaporate), or at reduced pressure (below 1 atm), e.g., below about 100 mm Hg. For example, the drying is carried out at atmospheric pressure and room temperature.

In one embodiment, Form C of 2-[4-(4-fluoro-benzyl)-piperidine-1-yl]-2-oxo-N-(2-oxo-2,3-dihydro-benzoxazol-6-yl)-acetamide is isolated in substantially pure form.

One skilled in the art will understand that the relative intensities and positions of the peaks obtained by X-ray powder diffraction and bands obtained by infrared or Raman spectroscopy may vary depending upon, inter alia, the sample preparation technique, the sample mounting procedure and the particular instrument employed.

### Compositions

In one embodiment, a method of treating conditions which require modulation of an NMDA receptor, e.g., an NR2B selective NMDA receptor, comprises administering an effective amount of Form C either alone as an active ingredient or as an additional ingredient of a pharmaceutically acceptable composition. The present invention also includes pharmaceutical compositions of Form C containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration effective for treating conditions which require modulation of an NMDA receptor, e.g., an NR2B selective NMDA receptor, may be accomplished using any route of administration for treating or lessening the severity of the disorder associated with modulation of an NMDA receptor. The exact amount will vary according to patient needs, depending on the age and general condition of the subject, the severity of the infection and the mode of administration, for example, orally, nasally, parenterally (subcutaneously, intravenously, intramuscularly, intrasternally and by infusion) by inhalation, rectally, vaginally, topically and by ocular administration.

Various solid oral dosage forms can be used for administering Form C including such solid forms as tablets, gelcaps, capsules, caplets, granules, lozenges and bulk powders. In such solid dosage forms Form C is mixed with at least one inert, pharmaceutically acceptable carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quarternary ammonium salts, g) wetting agents such as, for example cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Compositions suitable for buccal or sublingual administration include tablets, lozenges and pastilles, wherein the active ingredient is formulated with a carrier such as sugar and acacia, tragacanth, or gelatin and glycerin.

The solid dosage forms of tablets, capsules, pills and granules can be prepared with coatings and shells such as enteric coatings and other coatings well known in the pharmaceutical formulating art. They may optionally contain opacifying agents and can also be of a composition that they release the crystalline compound of the present invention. In another embodiment of the present invention, Form C can be formulated in a time release capsules, tablets and gels which is also advantageous in the targeted release of the crystalline compound of the present invention.

Various liquid oral dosage forms can also be used for administering Form C, including aqueous and non-aqueous solutions, emulsions, suspensions, syrups, and elixirs. In addition to the Form C, the liquid dosage forms may contain inert diluents commonly used in the art such as, for example, water or other solvents, solubilizing agents and emulsifiers, for example ethyl alcohol, ethyl carbonate, ethyl acetate, propylene glycol, oils, fatty acid esters and mixtures thereof. Besides inert diluents, the oral compositions can also include adjuvants such as wetting agents, emulsifying and suspending agents, sweetening, flavoring and perfuming agents. Aerosol formulations typically comprise typically comprise a solution or fine suspension of the crystalline compound of the present invention in physiologically acceptable aqueous or non-aqueous solvent and are usually presented in single or multidose quantitites in sterile form in a sealed container.

Injectable preparations of the present invention, for example, sterile injectable aqueous or oleaginous suspensions may be formulated according to the known art using suitable dispersing or wetting agents and suspending agents.

Suppositories for rectal administration of Form C can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the Form C can be administered by inhalation in the form of a powder (e.g., micronized) or in the form of atomized solutions or suspensions. The aerosol formulation can be placed into a pressurized acceptable propellant.

In one embodiment, the invention provides a composition comprising Form C of 2-[4-(4-fluoro-benzyl)-piperidine-1-yl]-2-oxo-N-(2-oxo-2,3-dihydro-benzoxazol-6-yl)-acetamide and a pharmaceutically acceptable carrier.

The invention also provides the use of Form C in the manufacture of a medicament for the treatment of conditions which require modulation of an NMDA receptor, e.g., an NR2B selective NMDA receptor.

In another embodiment, compositions of the present invention contain Form C between about 0.5% by weight and about 25%, between about 1% and about 20%, between about 2% and about 18%, between about 4% and about 15%, between about 6% and about 12%, between about 8% and about 10% by weight of the pharmaceutically acceptable composition.

The present invention further provides methods for treating conditions which require modulation of an NMDA receptor, e.g., an NR2B selective NMDA receptor, comprising administering an effective amount of Form C of 2-[4-(4-fluoro-benzyl)-piperidine-1-yl]-2-oxo-N-(2-oxo-2,3-dihydro-benzoxazol-6-yl)-acetamide.

Disorders which may be beneficially treated with NMDA antagonists include, for example, traumatic injury of brain [Neurol. Res., 21, 330-338 (1999)] or spinal cord [Eur. J. Pharmacol., 175, 165-74 (1990)], human immunodeficiency virus (HIV) related neuronal injury [Annu. Rev. Pharmacol. Toxicol., 1998; 38159-77], amyotrophic lateral sclerosis [Neurol. Res., 21, 309-12 (1999)], tolerance and/or dependence to opioid treatment of pain [Brain. Res., 731, 171-181 (1996)], withdrawal syndromes of e.g., alcohol, opioids or cocaine [Drug and Alcohol Depend., 59, 1-15 (2000)], muscular spasm [Neurosci. Lett., 73, 143-148 (1987)], dementia of various origins [Expert Opin. Investig. Drugs, 9, 1397-406 (2000)]. An NMDA antagonist may also be useful to treat cerebral ischemia of any origin (e.g., stroke, heart surgery), chronic neurodegenerative disorders, such as Alzheimer's disease, Parkinson's disease, Huntington's disease, pain (e.g., posttraumatic or postoperative) and chronic pain states, such as neuropathic pain or cancer related pain, epilepsy, anxiety, depression, migraine, psychosis, hypoglycemia, degenerative disorders of the retina (e.g., CMV retinitis), glaucoma, asthma, tinnitus, aminoglycoside antibiotic-induced hearing loss [Drug News Perspect 11, 523-569 (1998) and International Publication No. WO 00/00197].

In one embodiment of the present invention, the condition treated is pain and chronic pain states comprising administering an effective amount of Form C of 2-[4-(4-fluoro-benzyl)-piperidine-1-yl]-2-oxo-N-(2-oxo-2,3-dihydro-benzoxazol-6-yl)-acetamide.

In yet another embodiment, the condition represented by pain and chronic pain states is diabetic neuropathic pain (diabetic neuropathy). In another embodiment, the diabetic neuropathic pain is due to diabetes mellitus (e.g., type I or type II diabetes mellitus). In a further embodiment, the condition represented by pain and chronic pain is diabetic peripheral neuropathic pain (DPNP). In other embodiments, the condition represented by pain and chronic pain is diabetic autonomic neuropathic pain. In yet other embodiments, the condition represented by pain and chronic pain is diabetic proximal neuropathic pain. In other embodiments, the condition represented by pain and chronic pain states is diabetic focal neuropathic pain.

In yet other embodiments, the condition represented by pain and chronic pain states is neuralgias (e.g., post-herpetic neuralgia).

In another embodiment, Form C may be beneficially used for the treatment of traumatic injury of brain or spinal cord, human immunodeficiency virus (HIV) related neuronal injury, amyotrophic lateral sclerosis, tolerance and/or dependence to opioid treatment of pain, withdrawal syndromes of e.g., alcohol, opioids or cocaine, epilepsy, anxiety, depression, migraine, psychosis, muscular spasm, dementia of various origin, hypoglycemia, degenerative disorders of the retina, glaucoma, asthma, tinnitus, aminoglycoside antibiotic-induced hearing loss.

In another embodiment, the condition treated is schizophrenia, schizo-affective disorders, cognitive impairment accompanying schizophrenia, mild-to-moderate cognitive deficits comprising administering an effective amount of Form C of 2-[4-(4-fluoro-benzyl)-piperidine-1-yl]-2-oxo-N-(2-oxo-2,3-dihydro-benzoxazol-6-yl)-acetamide.

In another embodiment, the condition treated is bipolar disorder comprising administering an effective amount of Form C of 2-[4-(4-fluoro-benzyl)-piperidine-1-yl]-2-oxo-N-(2-oxo-2,3-dihydro-benzoxazol-6-yl)-acetamide.

In another embodiment, the condition treated is depression comprising administering an effective amount of Form C of 2-[4-(4-fluoro-benzyl)-piperidine-1-yl]-2-oxo-N-(2-oxo-2,3-dihydro-benzoxazol-6-yl)-acetamide.

In yet another embodiment, Form C can normally be administered in a daily dosage regimen (for an adult patient) of, for example, between about 0.01 mg and about 200 mg, between about 0.1 mg and about 150 mg, between about 10 mg and about 150 mg, between about 25 mg and 150 mg, between about 25 mg and 125 mg, between about 50 mg and 100 mg, inclusive of all ranges and sub-ranges therein.

In another embodiment, the active ingredient is administered in an amount of about 0.1 mg, about 0.5 mg, about 1 mg, about 2 mg, about 4 mg, about 8 mg, about 10 mg, about 15 mg, about 20 mg, about 25 mg, about 30.0 mg, about 35.0 mg, about 40.0 mg, about 45.0 mg, about 50.0 mg, about 55.0 mg, about 60.0 mg, about 65.0 mg, about 70.0 mg, about 80.0 mg, about 85.0 mg, about 90.0 mg, about 95.0 mg, about 100.0 mg, about 105.0 mg, about 110.0 mg, about 115.0 mg, or about 120.0 mg, inclusive of all ranges and sub-ranges therein.

In yet another embodiment, the active ingredient is administered in an amount of about 20 mg, about 40 mg, about 60 mg, or about 80 mg, inclusive of all ranges and sub-ranges therein.

In one embodiment, Form C can be administered 1 to 4 times per day, for example, once a day, twice a day. In another embodiment, Form C can suitably be administered for a period of continuous therapy, for example for a week or more.

In another embodiment, the disorders represented by diabetic neuropathic pain (diabetic neuropathy), diabetic neuropathic pain that is due to diabetes mellitus (e.g., type I or type II diabetes mellitus), diabetic peripheral neuropathic pain (DPNP), diabetic autonomic neuropathic pain, diabetic proximal neuropathic pain, diabetic focal neuropathic pain or neuralgias (e.g., post-herpetic neuralgia) is treated by administering an effective amount of Form C in the dosage amount from about 25 mg to about 125 mg to a patient in need thereof.

In yet another embodiment, the disorder represented by diabetic neuropathic pain (diabetic neuropathy), diabetic neuropathic pain that is due to diabetes mellitus (e.g., type I or type II diabetes mellitus), diabetic peripheral neuropathic pain (DPNP), diabetic autonomic neuropathic pain, diabetic proximal neuropathic pain, diabetic focal neuropathic pain or neuralgias (e.g., post-herpetic neuralgia) is treated by administering an effective amount of Form C in the dosage amount of about 20 mg, about 40 mg, about 60 mg, or about 80 mg, inclusive of all ranges and sub-ranges therein.

In another embodiment, the disorder represented by schizophrenia, schizo-affective disorders, cognitive impairment accompanying schizophrenia, mild-to-moderate cognitive deficits is treated by administering an effective amount of Form C in the dosage amount from about 25 mg to about 125 mg to a patient in need thereof.

In another embodiment, the disorder represented by schizophrenia, schizo-affective disorders, cognitive impairment accompanying schizophrenia, mild-to-moderate cognitive deficits is treated by administering an effective amount of Form C in the dosage amount of about 20 mg, about 40 mg, about 60 mg, or about 80 mg, inclusive of all ranges and sub-ranges therein.

In another embodiment, the disorder represented by bipolar disorder is treated by administering an effective amount of Form C in the dosage amount from about 25 mg to about 125 mg to a patient in need thereof.

In another embodiment, the disorder represented by bipolar disorder is treated by administering an effective amount of Form C in the dosage amount of about 20 mg, about 40 mg, about 60 mg, or about 80 mg, inclusive of all ranges and sub-ranges therein.

In another embodiment, the disorder represented by depression is treated by administering an effective amount of Form C in the dosage amount from about 25 mg to about 125 mg to a patient in need thereof.

In another embodiment, the disorder represented by depression is treated by administering an effective amount of Form C in the dosage amount about 20 mg, about 40 mg, about 60 mg, or about 80 mg, inclusive of all ranges and sub-ranges therein.

It will be understood that the specific dose level and frequency of dosage for any particular subject may be varied and will depend on a variety of factors including the activity of the specific compound employed, the metabolic stability and the length of action of that compound, the species, age, body weight, general health, sex and diet of the subject, the mode and time of administration, rate of excretion, drug combination, and severity of the particular combination.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The following examples are merely illustrative of the present invention and should not be construed as limiting the scope of the invention in any way as many variations and equivalents that are encompassed by the present invention will become apparent to those skilled in the art upon reading the present disclosure.

### EXAMPLES

### X-Ray Powder Diffractometry (XRD)

A small amount of sample was loaded on a zero background holder and exposed to CuKα radiation (30 kV×15 mA) having a wavelength λ of 1.541 Å in a wide-angle bench-top X-ray diffractometer (Model MiniFlex, Rigaku/MSC Inc., Woodlands, Tex.). The instrument was operated in the step-scan mode, in increments of 0.05°2. The angular range was 2 to 40° 2, and the scan rates ranged from 0.5-1°2. The data collection and analyses were performed with commercially available software (JADE, version 7.1, Materials Data, Inc., Livermore, Calif.).

### Fourier Transform Raman and IR Spectroscopy (FT-Raman and FT-IR)

For FT-Raman, a small amount of sample (LT 1 mg) was loaded on a glass slide and exposed to Raman laser in a Raman spectrophotometer (Thermo Nicolet Nexus 670 FT-IR/FT-Raman spectrometer, Thermo Electron, Waltham Mass.) using Nicolet EZ Omnic 5.1 software. All spectra were run at 3600-100 cm⁻¹ stokes shift, 300 scans and 2 cm⁻¹ resolution with laser output between 0.8 and 0.9 watts. For FT-IR, a small amount of sample (LT 1 mg) was loaded onto Durascope™ diamond stage an exposed to an IR beam in the FT-IR spectrometer using attenuated total diffuse reflectance (ATR) mode. All spectra were run at 4000-525 cm⁻¹ wavenumbers, 16 scans and 2 cm⁻¹ resolution.

### Differential Scanning Calorimetry (DSC)

A differential scanning calorimeter (MDSC Q1000, TA Instruments, New Castle, Del.) with a refrigerated cooling accessory was used. The instrument was calibrated with pure samples of indium. About 0.5-1 mg sample was weighed in open non-hermetic aluminum pans with 50 μm pierced cover lid and heated under dry nitrogen purge (flow rate 50 ml/min). The heating program was run under modulated conditions of ±1° C./60 amplitude at 2° C./min between 0 and 250° C. The data was analyzed using Universal Analysis 2000 (TA instruments, New Castle, Del.).

### Thermogravimetry

A Thermogravimetric analyzer (Pyris 1, Perkin Elmer, Wellesley, Mass.) with air cooling was used. About 0.5-1 mg sample was weighed in platinum TGA pans and heated under dry nitrogen purge (flow rate 70 ml/min) at 10° C./min. The data was analyzed using Pyris software (version 5.00.02, Perkin Elmer, Wellesley, Mass.).

### Synthesis of 2-[4-(4-Fluorobenzyl)-piperidin-1-yl]-2-oxo-N-(2-oxo-2,3-dihydro-benzoxazol-6-yl)-acetamide dihydrate

6-Amino-3H-benzoxazol-2-one (5.6 g, 0.037 mol) was added to a stirred mixture of [4-(4-fluorobenzyl)-piperidin-1-yl]-oxoacetic acid (11.8 g, 0.045 mol), O-benzotriazol-1-yl-N,N,N′,N′-tetramethyl-uronium hexafluoro-phosphate (HBTU) (16.85 g, 0.045 mol), triethylamine (4.55 g, 6.24 ml, 0.045 mol) and dimethylformamide (100 ml). The resulting solution was stirred for 2 hours at room temperature, then 8% NaHCO₃ solution (136 ml) was added dropwise. The mixture was stirred for a further 4 hours. The resulting crystals were filtered and washed twice with 150 ml of water. The wet product was dissolved in acetone (300 ml) and added dropwise to a mixture of 1% NaHCO₃ solution (200 ml) and acetone (80 ml) below 10° C. The resulting mixture was stirred for 1 hour, washed three times with water (70 ml) and dried at 50° C. to yield 8.5 g of 2-[4-(4-fluorobenzyl)-piperidin-1-yl]-2-oxo-N-(2-oxo-2,3-dihydro-benzoxazol-6-yl)-acetamide dihydrate. Water content (Karl-Fischer): 8.3%.

### Example 1

### Preparation of Form C of 2-[4-(4-fluoro-benzyl)-piperidine-1-yl]-2-oxo-N-(2-oxo-2,3-dihydro-benzoxazol-6-yl)-acetamide

Approximately 200 mg of 2-[4-(4-fluorobenzyl)-piperidin-1-yl]-2-oxo-N-(2-oxo-2,3-dihydro-benzoxazol-6-yl)-acetamide dihydrate was weighed into each of three glass scintillation vials. Approximately 5 mL of a 10/90, 20/80 and 30/70 water/acetone (v/v) solvent mixture was individually added to the glass vials (one solvent mixture per vial) and the resulting three mixtures were dispersed for 2 minutes by vortex. Each of the three vials was then capped and allowed to reach equilibrium at room temperature for 1 month. Each slurry was then carefully filtered and dried using a Whatman No. 4 cellulosic paper filter with a vacuum filtration flask apparatus. The product isolated from each vial was Form C of 2-[4-(4-fluoro-benzyl)-piperidine-1-yl]-2-oxo-N-(2-oxo-2,3-dihydro-benzoxazol-6-yl)-acetamide.

Peak positions for the XRPD pattern in FIG. 1 (Form C) are provided in Table 1.

TABLE 1
Form C

| 2θ (°) | d-spacing (\acute{Å}) |
|---|---|
| 6.4 | 13.9 |
| 8.0 | 11.1 |
| 9.6 | 9.2 |
| 11.5 | 7.7 |
| 12.8 | 6.9 |
| 13.7 | 6.5 |
| 15.4 | 5.8 |
| 16.0 | 5.5 |
| 17.0 | 5.2 |
| 17.3 | 5.1 |
| 18.9 | 4.7 |
| 19.3 | 4.6 |
| 19.8 | 4.5 |
| 20.4 | 4.3 |
| 21.2 | 4.2 |
| 21.7 | 4.1 |
| 24.1 | 3.7 |
| 24.6 | 3.6 |
| 25.8 | 3.5 |
| 26.3 | 3.4 |
| 27.1 | 3.3 |
| 27.5 | 3.2 |
| 28.7 | 3.1 |
| 29.0 | 3.1 |
| 29.7 | 3.0 |
| 30.5 | 2.9 |
| 32.4 | 2.8 |
| 32.8 | 2.7 |
| 33.3 | 2.7 |
| 33.5 | 2.7 |
| 34.7 | 2.6 |
| 35.3 | 2.5 |
| 35.7 | 2.5 |
| 36.7 | 2.4 |
| 37.3 | 2.4 |
| 38.5 | 2.3 |
| 39.3 | 2.3 |

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure.

The depicted and described embodiments of the invention are exemplary only, and are not exhaustive of the scope of the invention. Consequently, the invention is intended to be limited only by the spirit and scope of the appended claims, giving full cognizance to equivalence in all respects.

What is claimed is:

1. A crystalline form of 2-[4-(4-fluoro-benzyl)-piperidine-1-yl]-2-oxo-N-(2-oxo-2,3-dihydro-benzoxazole-6-yl)-acetamide as represented by formula (1) having an X-ray powder diffraction pattern comprising characteristic peaks at about 6.4, about 13.7, and about 25.8±0.2 degrees 2θ.

2. A crystalline form of 2-[4-(4-fluoro-benzyl)-piperidine-1-yl]-2-oxo-N-(2-oxo-2,3-dihydro-benzoxazole-6-yl)-acetamide having an infrared spectrum comprising characteristic absorption bands at about 3278, about 3106, about 2846, about 1683 and about 1560 cm⁻¹.

3. The crystalline form as in claim 1 or 2 having an X-ray diffraction pattern further comprising d spacing peaks at about 13.9, about 6.5, and about 3.5 Å.

4. A crystalline form of 2-[4-(4-fluoro-benzyl)-piperidine-1-yl]-2-oxo-N-(2-oxo-2,3-dihydro-benzoxazole-6-yl)-acetamide having a Raman spectrum comprising characteristic absorption bands at about 3280, about 3030, about 1730 and about 1570 cm⁻¹.

5. The crystalline form as in claim 1 or 4, further comprising d spacing peaks at about 13.9, about 6.5, and about 3.5 Å.

6. A process for preparing the crystalline form of claim 1, comprising:
(i) forming a mixture of 2-[4-(4-fluoro-benzyl)-piperidine-1-yl]-2-oxo-N-(2-oxo-2,3-dihydro-benzoxazole-6-yl)-acetamide dihydrate, water and acetone;
(ii) maintaining the mixture for a period of time, and
(iii) optionally isolating the crystalline form.

7. The process of claim 6, wherein the ratio of water to acetone is from about 10:90 to about 30:70 v/v.

8. The process of claim 6, wherein step (ii) comprises maintaining the mixture at room temperature.

9. The process of claim 6, wherein the period of time is about one month.

10. A pharmaceutical composition comprising the crystalline form as in claim 1.

11. A pharmaceutical composition according to claim 10 in the amount from about 25 mg to about 125 mg further comprising a pharmaceutically acceptable carrier.

12. The pharmaceutical composition according to claim 11, wherein the crystalline form is between about 0.5% and 25% by weight of the composition.

13. A method for treating and/or preventing a condition which requires modulation of an NMDA receptor comprising administering to a patient in need thereof, an effective amount of the crystalline form as in claim 1.

14. The method of claim 13, wherein the NMDA receptor is an NR2B selective NMDA receptor.

15. A method of treating a condition selected from the group consisting of pain and chronic pain states, schizophrenia, bipolar disorder, and depression comprising administering to a patient in need thereof a pharmaceutical composition according to claim 11.

16. The method of claim 15, wherein the condition is pain and chronic pain states.

17. The method of claim 15, wherein the condition is schizophrenia.

18. The method of claim 15, wherein the condition is bipolar disorder.

19. The method of claim 15, wherein the condition is depression.
